SEQUENCE LISTING (49 sequences)

SEQ ID NO: 1 - protein, 10 aa, Artificial Sequence
  model peptide of the tandem repeat decapeptide derived from foot protein 1 (FP-1, Mytilus edulis)
AKPSYPPTYK

SEQ ID NO: 2 - protein, 20 aa, Artificial Sequence
  2 times repeated sequence derived from foot protein 1 (FP-1, Mytilus edulis)
AKPSYPPTYKAKPSYPPTYK

SEQ ID NO: 3 - protein, 60 aa, Artificial Sequence
  6 times repeated sequence derived from foot protein 1 (FP-1, Mytilus edulis)
AKPSYPPTYKAKPSYPPTYKAKPSYPPTYKAKPSYPPTYKAKPSYPPTYKAKPSYPPTYK

SEQ ID NO: 4 - protein, 39 aa, Artificial Sequence
  partial sequence of foot protein type 2 (FP-2, Mytilus californianus)
EVHACKPNPCKNNGRCYPDGKTGYKCKCVGGYSGPTCAC

SEQ ID NO: 5 - protein, 52 aa, Artificial Sequence
  Foot protein type 3 (FP-3, Mytilus edulis)
ADYYGPKYGPPRRYGGGNYNRYGGSRRYGGYKGWNNGWKRGRWGRKYYEFEF

SEQ ID NO: 6 - protein, 46 aa, Artificial Sequence
  Foot protein type 3 (FP-3, Mytilus galloprovincialis  mgfp-3A)
ADYYGPKYGPPRRYGGGNYNRYGRRYGGYKGWNNGWKRGRWGRKYY

SEQ ID NO: 7 - protein, 60 aa, Artificial Sequence
  partial sequence from foot protein type 4 (Mytilus californianus)
GHVHRHRVLHKHVHNHRVLHKHLHKHQVLHGHVHRHQVLHKHVHNHRVLHKHLHKHQVLH

SEQ ID NO: 8 - protein, 75 aa, Artificial Sequence
  Foot protein type5 (FP-5, Mytilus edulis)
SSEEYKGGYYPGNAYHYHSGGSYHGSGYHGGYKGKYYGKAKKYYYKYKNSGKYKYLKKARKYHRKGYKKYYGGSS

SEQ ID NO: 9 - protein, 76 aa, Artificial Sequence
  Foot protein 5 (FP-5, Mytilus edulis)
SSEEYKGGYYPGNTYHYHSGGSYHGSGYHGGYKGKYYGKAKKYYYKYKNSGKYKYLKKARKYHRKGYKKYYGGGSS

SEQ ID NO: 10 - protein, 71 aa, Artificial Sequence
  Foot protein 5 (FP-5, Mytilus coruscus)
YDDYSDGYYPGSAYNYPSGSHWHGHGYKGKYYGKGKKYYYKFKRTGKYKYLKKARKYHRKGYKKHYGGSSS

SEQ ID NO: 11 - protein, 76 aa, Artificial Sequence; identical to SEQ ID NO: 9
  mussel adhesive protein foot protein type5 from (Mytilus galloprovincialis)

SEQ ID NO: 12 - protein, 99 aa, Artificial Sequence
  mussel adhesive protein foot protein type 6
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 13 - protein, 194 aa, Artificial Sequence
  hybrid mussel adhesive protein (FP-151, MEFP-5 based Kollodis)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 14 - protein, 196 aa, Artificial Sequence
  hybrid mussel adhesive protein (FP-151, MGFP-5 based)
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 15 - protein, 3 aa, Artificial Sequence
  Fibronectin-derived peptide (RGD)
RGD

SEQ ID NO: 16 - protein, 6 aa, Artificial Sequence
  Fibronectin-derived peptide (GRGDSP)
GRGDSP

SEQ ID NO: 17 - protein, 10 aa, Artificial Sequence
  Fibronectin-derived peptide (PHSRN-RGDSP)
PHSRNRGDSP

SEQ ID NO: 18 - protein, 9 aa, Artificial Sequence
  Fibronectin-derived peptide (SPPRRARVT)
SPPRRARVT

SEQ ID NO: 19 - protein, 8 aa, Artificial Sequence
  Fibronectin-derived peptide (WQPPRARI)
WQPPRARI

SEQ ID NO: 20 - protein, 15 aa, Artificial Sequence
  Fibronectin-derived peptide (KNNQKSEPLIGRKKT)
KNNQKSEPLIGRKKT

SEQ ID NO: 21 - protein, 12 aa, Artificial Sequence
  Laminin-derived peptide (RKRLQVQLSIRT)
RKRLQVQLSIRT

SEQ ID NO: 22 - protein, 12 aa, Artificial Sequence
  Laminin-derived peptide (GKNTGDHFVLYM)
GKNTGDHFVLYM

SEQ ID NO: 23 - protein, 13 aa, Artificial Sequence
  Laminin-derived peptide (VVSLYNFEQTFML)
VVSLYNFEQTFML

SEQ ID NO: 24 - protein, 12 aa, Artificial Sequence
  Laminin-derived peptide (RFDQELRLVSYN)
RFDQELRLVSYN

SEQ ID NO: 25 - protein, 13 aa, Artificial Sequence
  Laminin-derived peptide (RLVSYSGVLFFLK)
RLVSYSGVLFFLK

SEQ ID NO: 26 - protein, 12 aa, Artificial Sequence
  Laminin-derived peptide (ASKAIQVFLLGG)
ASKAIQVFLLGG

SEQ ID NO: 27 - protein, 12 aa, Artificial Sequence
  Laminin-derived peptide (VLVRVERATVFS)
THRPPMWSPVWP

SEQ ID NO: 28 - protein, 12 aa, Artificial Sequence
  Laminin-derived peptide (TVFSVDQDNMLE)
TVFSVDQDNMLE

SEQ ID NO: 29 - protein, 12 aa, Artificial Sequence
  Laminin-derived peptide (RLRGPQRVFDLH)
RLRGPQRVFDLH

SEQ ID NO: 30 - protein, 11 aa, Artificial Sequence
  Laminin-derived peptide (FDLHQNMGSVN)
FDLHQNMGSVN

SEQ ID NO: 31 - protein, 12 aa, Artificial Sequence
  Laminin-derived peptide (QQNLGSVNVSTG)
QQNLGSVNVSTG

SEQ ID NO: 32 - protein, 12 aa, Artificial Sequence
  Laminin-derived (SRATAQKVSRRS)
SRATAQKVSRRS

SEQ ID NO: 33 - protein, 16 aa, Artificial Sequence
  FGF-derived peptide (ANRYLAMKEDGRLLAS)
ANRYLAMKEDGRLLAS

SEQ ID NO: 34 - protein, 11 aa, Artificial Sequence
  FGF-derived peptide (HFKDPKRLYCK)
HFKDPKRLYCK

SEQ ID NO: 35 - protein, 8 aa, Artificial Sequence
  FGF-derived peptide (FLPMSAKS)
FLPMSAKS

SEQ ID NO: 36 - protein, 8 aa, Artificial Sequence
  FGF-derived peptide (KTGPGQKA)
KTGPGQKA

SEQ ID NO: 37 - protein, 12 aa, Artificial Sequence
  TGF-derived peptide (LTGKNFPMFHRN)
LTGKNFPMFHRN

SEQ ID NO: 38 - protein, 12 aa, Artificial Sequence
  TGF-derived peptide (MHRMPSFLPTTL)
MHRMPSFLPTTL

SEQ ID NO: 39 - protein, 20 aa, Artificial Sequence
  WNT-derived peptide (LCCGRGHRTRTQRVTERCNC)
LCCGRGHRTRTQRVTERCNC

SEQ ID NO: 40 - protein, 20 aa, Artificial Sequence
  WNT-derived peptide (LGTQGRLCNKTSEGMDGCEL)
LGTQGRLCNKTSEGMDGCEL

SEQ ID NO: 41 - protein, 10 aa, Artificial Sequence
  LIF-derived peptide (IVPLLLLVLH)
IVPLLLLVLH

SEQ ID NO: 42 - protein, 18 aa, Artificial Sequence
  LIF-derived peptide (YTAQGEPFPNNVEKLCAP)
YTAQGEPFPNNVEKLCAP

SEQ ID NO: 43 - protein, 6 aa, Artificial Sequence
  Fibronectin-derived peptide (KLDAPT)
KLDAPT

SEQ ID NO: 44 - protein, 9 aa, Artificial Sequence
  Fibronectin-derived peptide (EILDVPSTT)
EILDVPSTT

SEQ ID NO: 45 - protein, 12 aa, Artificial Sequence
  Laminin-derived peptide (TWYKIAFQRNRK)
TWYKIAFQRNRK

SEQ ID NO: 46 - protein, 12 aa, Artificial Sequence
  Laminin-derived peptide (NRWHSIYITRFG)
NRWHSIYITRFG

SEQ ID NO: 47 - protein, 11 aa, Artificial Sequence
  Fibronectin-derived peptide
PHSRNGRGDSP

SEQ ID NO: 48 - protein, 9 aa, Artificial Sequence
  Fibronectin-derived peptide
ATETTITIS

SEQ ID NO: 49 - protein, 19 aa, Artificial Sequence
  Fibronectin-derived peptide
YEKPGSPPREVVPRPRPGV

(19) United States
(12) Patent Application Publication
(10) Pub. No.: US 2016/0355780 A1
(43) Pub. Date: Dec. 8, 2016

(54) DEFINED THREE DIMENSIONAL MICROENVIRONMENT FOR CELL CULTURE

(71) Applicants: AMOGREENTECH CO., LTD., Gimpo-si (KR); Kollodis BioSciences, Inc., North Augusta, SC (US)

(72) Inventors: Chan Kim, Nam-gu (KR); Kyuwon Baek, Seoul (KR); Hui-Gwan Goo, Seoul (KR); Sangjae Lee, Seoul (KR); Bongjin Hong, Pohang-si (KR); Song Hee Koo, Seoul (KR); In Yong Seo, Seoul (KR); Seung Hoon Lee, Paju-si (KR); Ji Hyun Lee, Seo-gu (KR); Seonho Jang, Seoul (KR); Dong-Sik Seo, Nam-gu (KR)

(21) Appl. No.: 15/173,488

(22) Filed: Jun. 3, 2016

Related U.S. Application Data
(60) Provisional application No. 62/171,767, filed on Jun. 5, 2015.

(51) Int. Cl. C12N 5/00; D01F 1/10; D01F 6/66; D01F 6/18; D01F 6/32; C12N 5/0735; D01F 6/12

(52) U.S. Cl. CPC C12N 5/0062; C12N 5/0075; C12N 5/0606; D01F 1/10; D01F 6/12; D01F 6/18; D01F 6/32; D01F 6/66; C12N 2533/30; C12N 2535/10; C12N 2513/00; C12N 2501/998

(57) ABSTRACT

This disclosure provides a three-dimensional (3D) microenvironment presenting defined biochemical and physical cues that regulate cellular behavior and use of the microenvironment. A composition to form the 3D microenvironment is provided by combining one or more natural or synthetic polymeric materials and substrate proteins recombinantly or chemically functionalized with a variety of bioactive peptides such as extracellular matrix-derived or growth factor-derived peptides. The disclosure also provides for devices and methods for screening for optimal combinations of the bioactive motifs in order to create an extracellular microenvironment that can regulate specific cellular behavior such as cell growth, proliferation, migration or differentiation.

### CROSS-REFERENCE TO RELATED APPLICATION

This application claims the benefit of U.S. Provisional Patent Application Ser. No. 62/171,767, filed Jun. 5, 2015, the disclosure of which is hereby incorporated herein in its entirety by this reference.

### TECHNICAL FIELD

This disclosure relates to three-dimensional extracellular microenvironment for culturing valuable cells including stem cells such as pluripotent stem cells. More particularly, this disclosure provides methods and surfaces for culturing embryonic stem cells and other adult stem cells on a defined three-dimensional microenvironment.

### BACKGROUND

ESCs (Embryonic Stem Cells) or iPSs (induced pluripotent stem cells) are capable of differentiating into any cell type of the body while adult stem cells, such as mesenchymal stem cells, are more limited in their ability to differentiate into different lineages. Emerging evidence has shown that they have the ability to generate unrelated cell types via genetic reprogramming (see L. Bouwens, et al., “The use of stem cells for pancreatic regeneration in diabetes mellitus,” Nat. Rev. Endocrinol. 2013; 9(10):598-606; R. C. Addis, et al., “Induced regeneration—the progress and promise of reprogramming for heart repair,” Nat. Med. 2013; 19(7):829-836). The stem cells represent highly promising cell sources for numerous biomedical applications, such as cell replacement therapies, tissue and organ engineering, and pharmacology and toxicology screens. Stem cell maintenance, proliferation and expansion are important for the applications above.

Generally, a feeder composed of monolayers of inactivated fibroblast cells or reconstituted basement membrane such as MATRIGEL® (BD Biosciences) or GELTREX® (Life Technologies) are required to maintain self-renewal and pluripotentcy of embryonic stem cells or induced pluripotent stem cells (iPS) in media containing serum or serum replacement. These conditions include animal component and it has been reported that hESC cultured under these conditions acquired a non-human mammalian sialic acid, Neu5Gc (see U.S. Patent Publication No. U.S. 2011/0039333). These conventional substrates have significant lack of batch-to-batch or lot-to-lot consistency, resulting in experimental variability.

Various methods or surfaces have been developed to overcome the challenges described above. For example, a gelatin-coated surface in the presence of secreted factors from feeder cells, allowing the cells to be cultured in the absence of feeder cell layers (i.e., feeder-free). For example, feeder cell layers can be avoided through the use of “conditioned medium” (CM) (see, C. Xu, et al., “Feeder-free growth of undifferentiated human embryonic stem cells,” Nat. Biotechnol. 19:971-974 (2001)).

Additionally, current two-dimensional (2D)-based cell culture systems, which suffer from inherent heterogeneity and limited scalability and reproducibility, are emerging as a bottleneck for producing sufficient numbers of high-quality cells for downstream applications. An attractive approach for scaling up production is to move cell culture from 2D to 3D and, accordingly, several 3D suspension systems have been probed for hPSCs production: cell aggregates, cells on microcarriers, and cells in alginate microencapsulates.

A major challenge remains with the in vitro expansion and culture of self-renewable stem cells and the subsequent differentiation of these cells. Recently, several protocols have been reported that alter culturing conditions and other factors (e.g., medium and design of cell culture vessel) that support reliable amplification of immature and differentiated stem cells. However, challenges still exist in optimizing the wide variety of platforms capable of supporting cell therapy needs (from “Large-scale expansion of stem cells for therapy and screening”).

The self-renewal and pluripotency of murine ESCs (mESCs) and human ESCs (hESCs) are regulated by a combination of extrinsic and intrinsic factors. The factors regulate signaling pathway to control pluripotency transcription factors such as Oct4, Sox2, and Nanog (see J. A. Thomson, et al. (1998), “Embryonic stem cell lines derived from human blastocysts,” Science 282(5391):1145-1147).

The hESCs show activated Nodal/Activin, FGF and WNT pathways and have the potential for long-term maintenance in undifferentiated state and generation of three germ layer derivatives (Sato et al., 2004, Thomson et al., 1998, Xiao et al., 2006). FGF2 promotes self-renewal of hESCs by activating the PI3K/Akt activation to promote cell proliferation, growth, motility, and survival. WNTs (wingless-type MMTV integration site family members) proteins also play an important role in controlling ESC maintenance.

Stem cell resides in a specialized microenvironment, stem cell niche, that provides extracellular cues to allow stem cell survival and to maintain a balance between self-renewal and differentiation. Extracellular matrix (ECM) proteins that bind to mainly integrin are key components shaping the niche and maintaining stem cell homoeostasis. Integrin cross-talk with other receptors regulates signaling Erk 1/2, Akt, or SMAds responsible for preserving sternness. Integrins can potentiate signaling pathways in response to growth factors, cytokines such as IL-3 or TGF-beta, essential ligands cell self-renewal or pluripotency of hESCs (see M. F. Brizzi, et al., “Extracellular matrix, integrins, and growth factors as tailors of the stem cell niche,” 2012 Current Opinion in Cell Biology Volume 24, Issue 5, 645-651).

Extracellular microenvironments, defined by biochemical cues and physical or mechanical cues, are a deciding factor in a wide range of cellular processes including cell adhesion, proliferation, differentiation, and expression of phenotype-specific functions (see D. E. Discher, et al., Science 2009, 26:324 (5935):1673-7, and R. O. Hynes, Trends Cell Biol. 1999, 9(12):M33-7).

Most cells in tissues are surrounded on all sides by a complex set of extracellular matrix (ECM) proteins that are critical in guiding cell function. Cells bind to the ECM via specific cell surface receptors such as integrin receptors, and this binding serves as a biochemical cue that can directly affect cell function. In addition, the ECM acts as a modulator of biochemical and mechanical stimuli that are present in tissues. For example, ECM proteins can sequester and release growth factors, control the rate of nutrient supply, as well as control cell shape and transmit mechanical signals to the cell surface.

ECM and growth factor signaling environments are the important mechanisms for regulating cell fate. These microenvironmental stimuli are processed through combinatorial signaling pathways. The interactions between signaling pathways are critical in determining cell fate including stem cells (C. J. Flaim, et al., Stem Cells Dev. 2008, 17(1):29-39).

Complexities associated with native extracellular matrix proteins, including complex structural composition, purification, immunogenicity and pathogen transmission have driven the development of synthetic biofunctionals for use as 2D (two-dimensional) or 3D (three-dimensional) extracellular microenvironments in order to mimic the regulatory characteristics of natural ECMs and ECM-bound growth factors (M. P. Lutolf, et al., Nat. Biotechnol. 23(1):47-55 (2005); and K. Ogiwara, et al., Biotechnol. Lett. 27(20):1633-7 (2005)).

Many attempts have been made to create a synthetic 2D or 3D extracellular microenvironment by incorporating cell adhesion ligands into synthetic surfaces. Biologically derived or synthetic materials have been explored as an extracellular microenvironment to gain control over the material and, thus, over the cellular behavior they induced. One example is a cross-linkable hyaluronic acid, alginate or polyethylene glycol-based hydrogel with an RGD peptide motif grafted onto the polymer backbone. (Woerly et al., J. Neural Transplant. Plasticity, 1995, 5:245-255; Imen et al., Biofunctionals, 2006, 27, p. 3451-3458; U.S. Patent Publication No. 2006/0134050.)

U.S. Pat. No. 8,728,818 disclosed a defined surface that presents ECM-derived peptide motifs to activate integrin to support self-renewal and pluripotency of stem cell, and U.S. Pat. No. 9,006,394 disclosed a peptide-presenting surface to support long-term self-renewal of human embryonic stem cell. The peptides are heparin-binding domain from vitronectin, fibronectin or from bone sialoprotein. The surfaces in these patents require soluble factors such as FGF or ROCK inhibitor to support long-term self renewal and pluripotency of ESCs.

However, existing technologies have some limitations in generating a microenvironment that induces a combinatorial signal pathway by selectively, simultaneously or sequentially activating at least two different cell surface receptors in a precise manner, due to their lack of physical or biochemical attributes. In addition, various microenvironmental cues are often intertwined and cannot be individually controlled in existing technologies.

A biochemically and physically defined 3D microenvironment has been developed that mimics native extracellular microenvironments by presenting combinatorial receptor-ligand interactions with controlled physical cues including surface morphology and fiber diameter. The engineered 3D microenvironment can be used as an array of cell culture environments for screening of cell culture or tissue engineering environment by elucidating or regulating cellular behaviors, such as cell adhesion, migration, growth, proliferation or morphogenesis as evidenced in stem cell assays.

This disclosure provides a microenvironmentally defined surface that can promote signaling pathway to generate WNT/β-catenin, FGF/MEK, TGF-STAT, or LIF/STAT3 to support stem cell culture in serum- and feeder-free conditions.

### BRIEF SUMMARY

In one aspect, a 3D microenvironment surface that induces integrin signaling to promote signal pathway for self-renewal or proliferation of pluripotent stem cell in serum- or feeder-free conditions is provided. It is well known that integrin signaling involves Erk activation and self-renewal of embryonic stem cell is mediated signal through Ras-Raf-MEK-Erk cascade.

Simultaneous ligation of four integrin heterodimers (α5β1, α6β1, α9β1, and αvβ5) promoted self-renewal (see Seung Tae Lee, et al., “Engineering integrin signaling for promoting embryonic stem cell self-renewal in a precisely defined niche,” Biomaterials 31 (2010) 1219-1226). But in this disclosure, integrin activation of α5β1 binding motif alone or in combination with α6β1 binding motif is sufficient to generate the signaling for self-renewal and proliferation of pluripotent stem cell in the serum-free and feeder-free defined conditions.

In another aspect, a biochemically defined microenvironment that simultaneously or sequentially induces integrin and fibroblast growth factor receptors to promote signal pathway for self-renewal or proliferation of pluripotent stem cell in serum- or feeder-free conditions is provided.

In another aspect, a combinatorial microenvironment comprising a nanofibrous substrate having an average diameter of 100 to 2000 nm, wherein the nanofibrous surface presents extracellular matrix mimetic, growth factor mimetic, WNT mimetic, cytokines mimetic, LIF mimetic or its combination is provided.

In other aspects, there is provided a method of preparing a cell-culturing substrate including: providing a biochemically defined surface, wherein integrin-activating peptide motifs are coated to form a biochemically defined surface for cell culture in a defined condition; and the peptide motif can activate integrin α5β1- and/or α6 β1-integrins to simultaneously or sequentially generate integrin-mediated signaling.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 represents nanofibrous substrate as a synthetic 3D microenvironment. Each nanofiber has 200, 500 and 800 nm diameter.

FIG. 2 represents a synthetic nanofibrous microenvironment formed by electrospinning biofunctional composition. A combinatorial presentation of various peptide motifs immobilized on the nanofiber surface provides biochemical cues were surfaced on nanofibrous substrate.

FIG. 3 represents nanofiber having MAPTRIX® particles whose size is about 2 μm. Various peptide motif can be presented on the particle surface in order to control the fate of various cells including stem cells.

FIG. 4 represents the layout of synthetic microenvironment to screen an optimal extracellular microenvironment. The surface of each well presents integrin-binding motif.

FIGS. 5A and 5B represent the colony attachment of murine embryonic stem cells (mESC) seeded on various substrates. FIG. 5A represents the colony of mESC cultured on nanofibrous substrate and the colony of mESC on nanofiber having particles in FIG. 5B. The surface-presenting PHSRN-RGDSP (SEQ ID NO:17) that can activate integrin α5β1 to support self-renewal and proliferation of embryonic stem cell provided more favorable environment so that the size of colony is larger than in other surfaces.

### DETAILED DESCRIPTION

This disclosure is directed to engineered extracellular microenvironments that mimic biochemically and/or physically natural extracellular microenvironments.

This disclosure also provides biochemically and physically defined 3D microenvironments surface that regulates cell surface receptors specifically, selectively, simultaneously, or sequentially to support self-renewal and pluripotency of embryonic stem cells.

As used herein, “microenvironment” refers to physical and/or biochemical cues, surrounding a cell in an organism or in the laboratory. Molecules, including small molecules such as compounds and soluble factors, macromolecules such as insoluble polymers, nutrients, growth factors, fluids, cytokines and parameters such as pH, ionic strength and gas composition, and the like surrounding the cell are the biochemical cues. The molecules for biochemical cues may be, reversibly or irreversibly in response to biological or physiological conditions, immobilized to the substrate.

A microenvironmentally, namely biochemically and physically, defined cell-culturing substrate is provided for self-renewal and pluripotency of stem cells in serum-free and feeder-free conditions for extended periods of time in culture. The microenvironmentally defined culture surface of this disclosure promotes more efficient attachment and expansion of pluripotent stem cells such as embryonic stem cells, as well as mesenchymal or neural stem cells in an undifferentiated state, as compared to standard culture substrates such as tissue culture-treated or serum-coated surfaces. In some embodiments, murine ES cells may be expanded on the microenvironmentally defined cell culture surface.

A microenvironmentally defined 3D surface is provided. The 3D surface may be microenvironmentally defined over media found within a cell culture plate or other structure. A substrate for the defined surface may include patterned or porous nanofiber being composed of various materials including polyvinylidene (PVDF), but not limited to cellulose, nylon, glass fiber, materials for bio-reactors used in batch or continuous cell culture or in bioreactors.

As used herein, “nanofiber” refers to the electroprocessed composition that may include particles being larger than a nanofiber as a result of the electroprocessed composition, where the surface of the nanofiber presents biochemical cues. Collectively, the nanofiber may provide in vivo-like microenvironment to regulate the fate of cells of interest.

This disclosure provides an electroprocessable biofunctional composition to engineer an extracellular microenvironment presenting controlled physical and/or biochemical cues. As used herein, “biofunctional composition” refers to a composition that comprises a bioactive component and a structural component that is electroprocessable polymer solution. An electroprocess including electrospinning or electro-spraying is a means of producing fibers or particles with diameters generally between 10 to 2,000 nanometers. It has the ability of producing fibers or particles that are far smaller than those produced by conventional means, such as wet spinning or melt spinning.

A bioactive component is a natural or synthetic polymer or protein-containing peptide motif As used herein, “peptide motif” refers to a short peptide, preferably three (3) to one hundred (100) amino acids in length that possesses a peptide derived from natural protein such as extracellular matrix (ECM) or growth factor that mimic natural ECM or GF activity. Preferably, the bioactive peptide is a peptide that was originally identified in nature, produced by an animal, plant, fungus or bacterium as part of their natural mechanism.

ECM and growth factor signaling environments are the important mechanisms for regulating cell fate and these microenvironmental stimuli are processed through combinatorial signaling pathways. The interactions between signaling pathways are critical in determining cell fate (C. J. Flaim, et al., Stem Cells Dev. 2008, 17(1):29-39).

The biochemically defined, peptide motif-presenting surfaces described herein are useful in a variety of contexts and applications. For example, the surfaces can be used for maintaining pluripotent cells in an undifferentiated state. In addition, the surfaces can be used for expanding a population of pluripotent cells without loss of differentiation potential. The biochemically defined, peptide-presenting surfaces are also useful for culturing pluripotent cells that are subsequently induced to differentiate by, for example, adding one or more differentiation agent to the media. Differentiated cells derived from pluripotent cells can be maintained on the biochemically defined surfaces.

Suitable pluripotent cells for use herein include ESCs and iPS cells, which preferably are from a primate, especially a human primate. As used herein, “embryonic stem cells” or “ESCs” mean a pluripotent cell or population of pluripotent cells derived from an inner cell mass of a blastocyst.

Regardless of the pluripotent cell used, the biochemically defined surfaces described herein can be constructed according to known methods. For example, one can use contact spotting of peptides onto glyoxylyl-functionalized glass slides (see, e.g., J. Falsey, et al., “Peptide and small molecule microarray for high throughput cell adhesion and functional assays,” Bioconjug. Chem. 12, 346-353 (2001)); contact printing of peptides onto acrylamide-coated glass slides; and spotting combinations of peptides onto a glass slide followed by in situ polymerization (see, e.g., Anderson et al., “Nanoliter-scale synthesis of arrayed biomaterials and application to human embryonic stem cells,” Nat. Biotechnol. 22:863 (2004)). In addition, one can use streptavidin-coated plates treated with a biotinylated peptide of interest or even polyacrylamide gels cross-linked to a peptide of interest. See, e.g., Klein et al., “Cell adhesion, cellular tension, and cell cycle control,” Meth. Enzymol. 426:155 (2007). Water-insoluble synthetic or natural hydrogels are also contemplated as providing a suitable peptide-presenting surface.

This disclosure provides an extracellular microenvironment comprised of a nanofiber presenting at least one or more extracellular matrix (ECM)-derived or growth factor (GF)-derived peptide motifs that precisely regulate cellular behavior such as cell adhesion, migration, growth or differentiation. The nanofiber may include dispersed particles being at least partially embedded into the nanofibers as a result of electroprocessed composition, the particle being larger than the average diameter of nanofibers.

The disclosure provides an electrospinnable biofunctional composition for a fibrous extracellular microenvironment comprised of two components, extracellular component and a structural component. In one embodiment, a structural component is a polymer to provide physical or mechanical cues such as pore size or elasticity, whereas extracellular component provides biochemical cues.

Any electrospinnable polymer, natural or synthetic, for use in this disclosure can be a structural component. Preferably, an electrospinnable polymer is a synthetic polymer that has the appropriate viscosity in solution. Any polymer meeting the above requirements is useful herein, and the selection of the specific polymer and acquisitions or preparation of such polymer would be conventionally practiced in the art (see reference here). Preferred for such electrospinnable polymers are selected from groups comprising polyvinylidene fluoride (PVDF), polyacrylonitrile (PAN), polyethersulfone (PES), polylactic acid (PLA), polyglycolic acid (PGA), poly (lactide-glycolic) acid (PLGA), polycaprolactone, poly(alkylene oxides) particularly poly(ethylene glycols), poly(vinyl alcohols), polypeptides, poly(amino acids), such as poly(lysine), poly(allylamines) (PAM), poly(acrylates), polyesters, polyphosphazenes, pluronic polyols, polyoxamers, poly(uronic acids) and copolymers, including graft polymers thereof.

In another aspect, the disclosure provides a nanofibrous matrix to mimic a natural extracellular microenvironment, wherein the matrix has an elastic or linear modulus of about 0.5 kPa to about 1 MPa and fiber diameter of about 100 nm to 1,000 nm. In one embodiment, the method of making the nanofibrous matrix includes (a) generating an electrostatic field between a first electrode and a second electrode, and (b) electrospinning a solution of biofunctional composition comprising the mussel adhesive protein and a synthetic polymer onto a collection surface located between the first electrode and the second electrode to provide a plurality of nanofibers on the collection surface.

The polymer may be selected to have a wide range of molecular weights, generally from as low as 100,000 up to millions of Daltons. Preferably, the selected polymer has a molecular weight of less than about 300,000 to 500,000.

In another embodiment, a hydrophilic polymer is used to form an electrospinnable biofunctional composition wherein polyethylene oxide or polyethylene glycol has a molecular weight of from about 30 kDa to about 300 kDa. In one embodiment, the fiber includes about 30 weight percent mussel adhesive protein and about 70 weight percent polyethylene oxide. In another embodiment, the fiber includes about 1 weight percent mussel adhesive protein and about 70 weight percent polyethylene glycol.

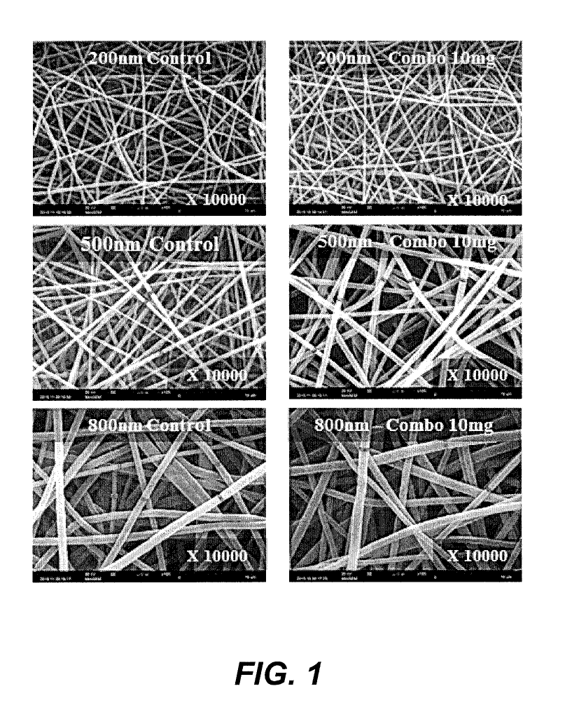

FIG. 1 shows scanning electron microscopy (SEM) images of nanofibers spun from solutions with PEO and PEG, respectively. Solutions with mussel adhesive protein/PEO ratios in the range 65:35-90:10 yielded cylindrical nanofibers with a mean diameter of approximately 500 nm and a relatively narrow size distribution.

In another embodiment, a hydrophobic polymer is used to form an electrospinnable biofunctional composition wherein PVDF, PAN, and/or PES, single or in combination, has a molecular weight of from about 50 Kda to about 500 kDa. In one embodiment, the nanofiber includes about 0.1 weight percent mussel adhesive protein and about 10 weight percent hydrophobic polymer.

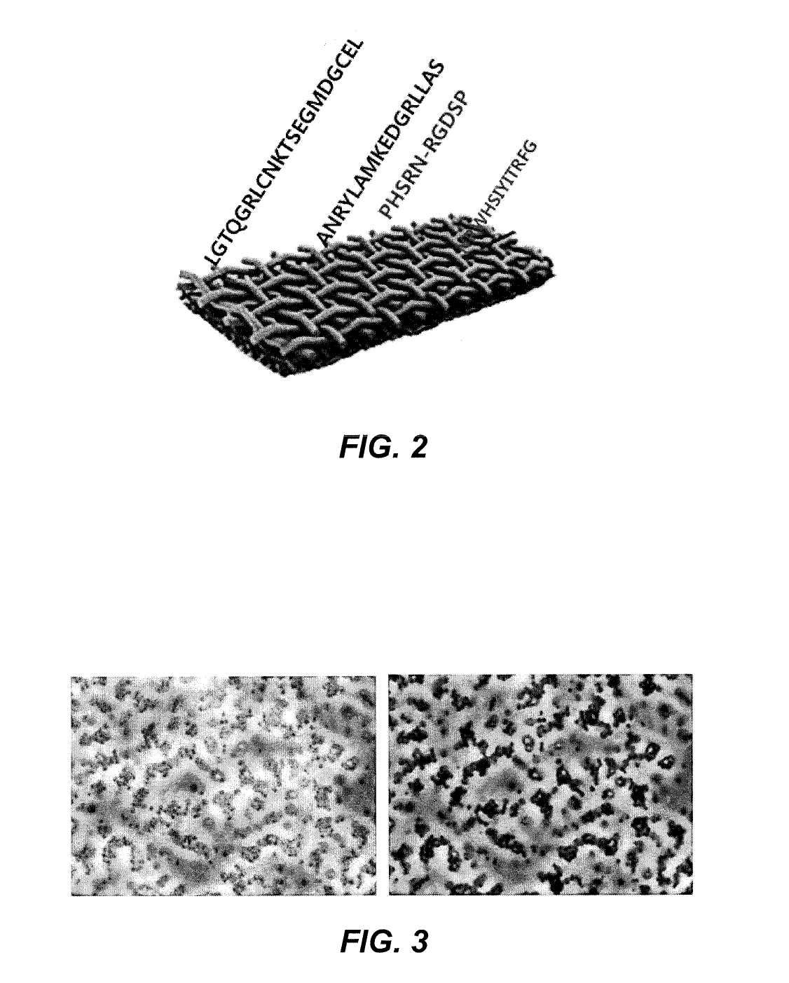

FIG. 2 is a scanning electron microscopy (SEM) image of nanofibers spun from solutions with mixture of PVDF and mussel adhesive protein. Solutions with mussel adhesive protein/PVDF ratios in the range 0.1:99.9-1:99 yielded cylindrical nanofibers with a mean diameter of approximately 500 nm and a relatively narrow size distribution.

Pore size of a matrix can affect cell behavior within the matrix and subtle changes in pore size can have a significant effect on cell behavior such as cell migration. If the pores become too large, the mechanical properties of the scaffold will be compromised due to void volume and as pore size increases further, the specific surface area will eventually reduce to a level that will limit cell adhesion.

The nanofiber of the disclosure can be enzymatically, ionically, covalently, or hydrogen bond mediated cross-linked to maintain its structural integrity in response to biological environments. The nanofiber of the disclosure may be cross-linked with an ionic or covalent cross-linking agent. Suitable ionic cross-linking agents include bivalent metal ions such as Ca2+, Ba2+, or Sr2+. Suitable covalent cross-linking agents include bifunctional cross-linking agents reactive toward amine and/or carboxylic acid groups of mussel adhesive protein. Representative covalent cross-linking agents include carbodiimides, allyl halide oxides, dialdehydes, diamines, and diisocyanates. In certain embodiments, the covalent cross-linking agent is selected from gluteraldehyde, hexamethylene diisocyanate, adipic acid hydrazide, and 1-ethyl-3-(3-dimethylaminopropyl) carbodiimide hydrochloride, epichlorohydrin, n-hydroxysuccinimide (NHS). Suitable enzyme for this disclosure is a transglutaminase or tyrosinase.

This disclosure provides a cell or tissue culturing system comprising a plurality of microenvironments comprising a composition that supports growth and self renewal of a stem cell by regulating at least two or more cell surface receptors. It has been known that cross-talk between integrins and growth factor receptors by two mechanism, i) two separate signals merge with one another in multiple levels inside the cells (see Legate, et al., “Genetic and cell biological analysis of integrin outside-in signaling,” Genes Dev. 2009, 23:397-418), or ii) FGF1 directly binds to integrin αvβ3 and induces the FGFR1-FGF1-integrin αvβ3 ternary complex (S. Mori, et al., “Direct binding of integrin αvβ3 to FGF1 plays a role in FGF1 signaling,” J. Biol. Chem. 2008, 283:18066-18075). In one embodiment, the extracellular microenvironment surface to activate integrin α5β1 and integrin 60 6β1 mediated signaling at the same time and FGFR to support self-renewal of pluripotent or multi-potent stem cell.

An extracellular component such as ECM protein or growth factors can be a natural or recombinant extracellular matrix protein, ECM-derived domain including core motif that binds to specific integrin or its mimetic, growth factor, GF-derived domain containing core motif that bind to specific binding sites of such growth factor receptor, or its mimetic. The mimetic comprises a recombinant protein or polypeptide functionalized with at least one or more peptide motifs derived from a variety of extracellular matrix proteins or growth factors.

Any suitable natural extracellular matrix proteins including, but not limited to, fibronectin, laminin, vitronectin, may be used as an extracellular component to activate integrins. Preferably, the extracellular matrix protein is fibronectin. More preferably, the fibronectin can be used alone or in combination with laminin, vitronectin or cadherin.

Any suitable natural growth factors are fibroblast growth factor (FGF) or transforming growth factor (TGF) may be used as an extracellular component to activate such growth factor receptors. Preferably, the growth factor can be used alone or in combination with FGF and TGF.

Generally, any extracellular mimetic component including extracellular matrix mimetic or growth factor mimetic comprises a substrate protein recombinantly or chemically functionalized with peptide motif derived from extracellular matrix proteins or growth factors.

Any suitable substrate protein including, but not limited to, fibrin, elastin, mussel adhesive protein may be used as the substrate protein to present extracellular component. Preferably, the protein is a recombinant mussel adhesive protein.

Any suitable recombinant mussel adhesive protein may be used as the extracellular component in this disclosure. Examples of commercially available substrate proteins include MAPTRIX® ECM marketed by Kollodis BioSciences, Inc. (North Augusta, S.C.). An optional third component is a biocompatible polymer (e.g., polyethylene glycol or polyvinylalcohol), which may be added to the compositions to enhance their physicomechanical characteristics such as physical or mechanical properties of a customizable microenvironment.

The MAPTRIX®, developed by Kollodis BioSciences Inc. (North Augusta, S.C.), are predesigned mussel adhesive protein or barnacle-based extracellular component mimetics. The mussel adhesive proteins were recombinantly functionalized with a variety of ECMs-, GFs-, or other ligand-derived peptides in order to mimic the bioactivity of naturally occurring ligands such as ECMs, GFs, or other soluble factors such as cytokines including IL-3 or LIF, which were demonstrated to have a similar bioactivity to natural or recombinant ECMs, GFs, or soluble factors in primary cell cultures as compared to various natural or recombinant ECM, GF or cytokine proteins. The pre-designed MAPTRIX® mimetics are highly advantageous for creating extracellular microenvironments. For example, it provides for the design of cell-specific or user-defined regulation of extracellular microenvironments to emulate the native microenvironment in terms of biochemical cues.

The MAPTRIX® is a fusion protein comprising a first peptide of mussel foot protein FP-5 that is selected from the group consisting of SEQ ID NOS:1-4, or barnacle-derived adhesive protein consisting of SEQ ID NO:5 and a second peptide of at least one selected from the group consisting of mussel FP-1 selected from the group consisting of SEQ ID NOS:6-8, mussel FP-2 (SEQ ID NO:9), mussel FP-3 selected from the group consisting of SEQ ID NOS:10-11, mussel FP-4 (SEQ ID NO:12), mussel FP-6 (SEQ ID NO:13) and fragment thereof, and the second peptide is linked to C-terminus, N-terminus or C- and N-terminus of the FP-5. Preferably, the second peptide is The FP-1 comprising an amino acid sequence of SEQ ID NO:6.

Extracellular components including integrin binding motif or growth factor receptor binding motif such as fibroblast growth factor (FGF) and transforming growth factor (TGF)-derived peptide motif, WNT and/or LIF (leukemia inhibitor factor) may also be incorporated into the mussel adhesive protein to further enhance the beneficial effect of the extracellular environment mimic on self-renewal and pluripotency of a stem cell.

There are 24 known integrin heterodimers comprised of one of 18 a subunits and one of 8 β subunits and these have a diverse range of functions mediating cell-cell adhesion, growth factor receptor responses and intracellular signaling cascades for cell migration, differentiation, survival and proliferation. A number of ECM molecules or domains are capable of assisting in the maintenance of undifferentiated hESC alone or in combination, including laminin 511 (see T. Miyazaki, et al., “Recombinant human laminin isoforms can support the undifferentiated growth of human embryonic stem cells,” Biochem. Biophys. Res. Commun., 375 (2008), pp. 27-32), fibronectin and vitronectin (see Melkoumian et al., “Synthetic peptide-acrylate surfaces for long-term self-renewal and cardiomyocyte differentiation of human embryonic stem cells,” Nat. Biotechnol. 28 (2010), pp. 606-610; Braam et al., “Recombinant vitronectin is a functionally defined substrate that supports human embryonic stem cell self-renewal via alphavbeta5 integrin,” Stem Cells, 26 (2008), 2257-2265)).

The extracellular domain of integrins can bind ECM proteins used in hESC support such as collagen, fibronectin, laminin and vitronectin as well as members of the SIBLING family (Small Integrin Binding Ligand, N-Linked Glycoproteins, e.g., osteopontin and bone sialoprotein). Integrin clustering occurs after ECM adhesion promoting lateral association with other cell surface receptors and increases in the cytoplasmic concentration of cell signaling molecules such as PI3-kinase and MEK-ERK, which are involved in hESC maintenance (see J. Li, et al., “MEK/ERK signaling contributes to the maintenance of human embryonic stem cell self-renewal,” Differentiation 75 (2007), 299-307).

Recently, the Hubbell laboratory developed and tested various synthetic substrates for their capacity to maintain mouse ES cell self-renewal and concluded that simultaneous ligation of α5β1-, αvβ5-, α6β1, and α9β1 integrins promotes sternness of ES cells. These integrins have also been implicated in the regulation of mouse and human ES cell self-renewal in a number of other studies performed under various growth conditions (see Sandhanakrishnan Cattavarayan, et al., α6β1- and αv-integrins are required long-term self-renewal of murine embryonic stern cells in the absence of LIF, BMC Cell Biology 2015, 16:3; Y. Meng, et al., “Characterization of integrin engagement during defined human embryonic stem cell culture,” FASEB J. 2010; 24(4):1056-65; S. R. Braam, et al.. “Recombinant vitronectin is a functionally defined substrate that supports human embryonic stern cell self-renewal via αvβ5 integrin,” Stem Cells 2008; 26(9):2257-65).

This disclosure also provides a microenvironmentally defined 3D surface that activates α5β1, α6β1 and/or αvβ5 simultaneously or sequentially in order to regulate signaling pathway for self-renewal and pluripotency maintenance of a stem cell. Any suitable substrate protein containing peptide ligand to activate integrin α5β1-, αvβ5-, α6β1, or α9β1 simultaneously or sequentially to support self-renewal and pluripotency of a stem cell. In one embodiment, the microenvironment surface provides a substrate protein presenting 60 5β1 integrin activating motif or heparin binding motif derived from fibronectin domain III. Any suitable α5β1 integrin activating- or heparin binding motif can be selected from RGD (SEQ ID NO:15), GRGDSP (SEQ ID NO:16), PHSRN-RGDSP (SEQ ID NO:17), SPPRRARVT (SEQ ID NO:18), WQPPRARI (SEQ ID NO:19), KNNQKSEPLIGRKKT (SEQ ID NO:20), or its combination of a5131 integrin activating motif and heparin binding motif.

In another embodiment, the microenvironment surface provides a substrate protein presenting α6β1 integrin activating motif-derived laminin al or laminin α5 LG domain to support self-renewal and pluripotency of a stem cell. Any suitable α6β1 integrin activating motif can be selected from GKNTGDHFVLYM (SEQ ID NO:22), VVSLYNFEQTFML (SEQ ID NO:23), RFDQELRLVSYN (SEQ ID NO:24), RLVSYSGVLFFLK (SEQ ID NO:25), ASKAIQVFLLGG (SEQ ID NO:26), VLVRVERATVFS (SEQ ID NO:27), TVFSVDQDNMLE (SEQ ID NO:28), RLRGPQRVFDLH (SEQ ID NO:29), FDLHQNMGSVN (SEQ ID NO:30), QQNLGSVNVSTG (SEQ ID NO:31), SRATAQKVSRRS (SEQ ID NO:32), TWYKIAFQRNRK (SEQ ID NO:45), or NRWHSIYITRFG (SEQ ID NO:46).

In another embodiment, the 3D microenvironment surface provides a substrate protein presenting a combinatorial motif of α5β1 integrin activating motif and α6β1 binding motif at the same time to support self-renewal and pluripotency of a stem cell. Suitable combinatorial motif is a combination of PHSRN-RGDSP (SEQ ID NO:17) and NRWHSIYITRFG (SEQ ID NO:46) to support self-renewal and pluripotency of a stem cell.

Fibroblast growth factors (FGFs) are essential for maintaining self-renewal in human embryonic stem cells and induced pluripotent stem cells. Recombinant basic FGF (bFGF or FGF2) is conventionally used to culture pluripotent stem cells. Today, FGF family consists of 23 members including acidic and basic fibroblast growth factor, and each FGF has canofin, hexfin, and decafin domain (S. Li, et al., “Fibroblast growth factor-derived peptides: functional agonists of the fibroblast growth factor receptor,” J. Neurochem. 2008 Feb. 104(3):667-82; S. Li, et al., “Agonists of fibroblast growth factor receptor induce neurite outgrowth and survival of cerebellar granule neurons,” Dev. Neurobiol. 2009, 69(13):837-54; Li Shizhong, et al., “Neuritogenic and Neuroprotective Properties of Peptide Agonists of the Fibroblast Growth Factor Receptor,” Int. J. Mol. Sci. 2010, 11(6):2291-2305).

FGFRs are transmembrane glycoproteins with three extracellular domains, Ig1, Ig2 and Ig3. An FGFR fragment Ig2 and Ig3 is the minimal unit sufficient for specific ligand binding (see V. Manfè, et al., “Peptides derived from specific interaction sites of the FGF 2-FGF receptor complexes induce receptor activation and signaling,” J. Neurochem. 2010, 114(1):74-86; S. K. Olsen, et al. (2004), “Insights into the molecular basis for fibroblast growth factor receptor autoinhibition and ligand-binding promiscuity,” Proc. Natl. Acad. Sci. USA 101 935-940).

Bell et al. (see, “Rotational coupling of the transmembrane and kinase domains of the Neu receptor tyrosine kinase,” Mol. Biol. Cell 11:3589-3599 (2000)) demonstrated that activation of receptor tyrosine kinases requires specific orientations of the kinase domains in a formed receptor dimer. The ligand binding mediates the optimal rotational positioning of the individual monomers within the dimer and thus the specific orientation of the catalytic domains. Binding of different agonists, such as FGF2 and canofins resulted in different modes of orientation of catalytic domains yielding differences in receptor activation (see V. Manfe, et al., “Peptides derived from specific interaction sites of the fibroblast growth factor 2—FGF receptor complexes induce receptor activation and signaling,” J. Neurochem. 2010; 114(1):74-86).

When a growth factor binds to the extracellular domain of a receptor tyrosine kinase (RTK), its dimerization is triggered with other adjacent RTKs. Dimerization leads to a rapid activation of the protein's cytoplasmic kinase domains and the activated receptor as a result then becomes autophosphorylated on multiple specific intracellular tyrosine residues, resulting in signal transduction cascade.

Recent studies have demonstrated that the immobilization of soluble factors such as FGF, TGF or cytokines to the ECM plays an important role in mediating their biological effects (see C. C. Rider (2006) “Heparin/heparan sulphate binding in the TGF-beta cytokine superfamily,” Biochem. Soc. Trans. 34:458-460). Presentation of soluble factors in an immobilized fashion alters their local effective concentration, bioavailability, and stability, and thereby modulates their effects on target cells. For example, NSC-proliferative regions in the SVZ are situated in proximity to regions, in which growth factors including basic fibroblast growth factor-2 are concentrated by heparan sulfate proteoglycan (HSPG) (see F. Mercier, et al. (2002), “Anatomy of the brain neurogenic zones revisited: fractones and the fibroblast/macrophage network,” J. Comp. Neurol. 451:170-188).

This disclosure provides the FGF mimetic comprising recombinant mussel adhesive protein functionalized with FGF-derived peptide motif derived from hexafin domain or canofin domain. Preferably, FGF mimetic peptide motif can be selected from hexafin domain-derived ANRYLAMKEDGRLLAS (SEQ ID NO:33) or canofin domain-derived HFKDPKRLYCK (SEQ ID NO:34), FLPMSAKS (SEQ ID NO:35), KTGPGQKAIL (SEQ ID NO:36).

In one embodiment of this disclosure, a 3D microenvironment surface that combinatorially regulates the activity of both integrin and growth factor receptor to support self-renewal and pluripotency of murine embryonic stem cell is provided. The microenvironment surface comprises a substrate protein functionalized with a peptide such as fibronectin-derived peptide PHSRN-GRGDSP (SEQ ID NO:47) to target α5β1 and FGF2-derived peptide ANRYLAMKEDGRLLAS (SEQ ID NO: 33) to target FGF receptor; FGFR2IIIc.

The present disclosure also provides a 3D microenvironment surface to activate TGF receptor or Frizzle receptor to induce signaling pathway to activate transcriptional factors for self-renewal and pluripotency of pluripotent stem cell. A recombinant mussel adhesive protein as a substrate protein containing TGF mimetic peptide to bind to TGFβ receptor domain TβRI or TβRII can be used in this disclosure. Preferably, TGFβ mimetic peptide can be selected from LTGKNFPMFHRN (SEQ ID NO:37) or MHRMPSFLPTTL (SEQ ID NO:38).

In one embodiment of this disclosure, a 3D microenvironment surface that combinatorially regulates the activity of both integrin and growth factor receptor to support self-renewal and pluripotency of an embryonic stem cell is provided. The microenvironment surface comprises a substrate protein presenting a combinatorial motif to activate α5β1 integrin and TGFβ receptor at the same time. The combinatorial motif is a combination of the substrate protein functionalized with a peptide such as fibronectin-derived peptide PHSRN-GRGDSP (SEQ ID NO:47) to target α5β1 and TGFβ-derived peptide LTGKNFPMFHRN (SEQ ID NO:37), or MHRMPSFLPTTL (SEQ ID NO:38).

This disclosure provides a 3D microenvironment surface that generates WNT/β-catenin signaling pathway by presenting WNT 1 peptide motif LCCGRGHRTRTQRVTERCNC (SEQ ID NO:39) or LGTQGRLCNKTSEGMDGCEL (SEQ ID NO:40). In one embodiment of the disclosure, a microenvironment surface that combinatorially regulates the activity of both integrin and frizzled receptor to support self-renewal and pluripotency of an embryonic stem cell is provided. The microenvironment surface comprises a substrate protein presenting a combinatorial motif to activate α5β1 integrin and frizzled receptor at the same time. The combinatorial motif is a combination of the substrate protein functionalized with a peptide such as fibronectin-derived peptide PHSRN-GRGDSP (SEQ ID NO:47) to target α5β1 and WNT-derived peptide LCCGRGHRTRTQRVTERCNC (SEQ ID NO:39) or LGTQGRLCNKTSEGMDGCEL (SEQ ID NO:40).

This disclosure provides a 3D microenvironment surface that generates LIF/STAT3 signaling pathway by presenting LIF peptide motif IVPLLLLVLH (SEQ ID NO:41) or YTAQGEPFPNNVEKLCAP (SEQ ID NO:42).

Various studies suggest that co-clustering or synergism occurs between downstream signaling molecules, once the basic requirements are met: growth factor receptor ligand-binding, integrin occupancy by a ligand and clustering of each type of receptor (see M. A. Schwartz and V. Baron, “Interactions between mitogenic stimuli, or, a thousand and one connections,” Curr. Opin. Cell Biol. 11:197-202 (1999); K. M. Yamada and E. H. J. Danen, “Integrin signaling” in Signaling Networks and Cell Cycle Control (ed. J. S. Gutkind) 1-25 (Humana Press, Totowa, N.J., 2000); S. Miyamoto, et al., “Integrins can collaborate with growth factors for phosphorylation of receptor tyrosine kinases and MAP kinase activation: roles of integrin aggregation and occupancy of receptors,” J. Cell Biol. 135:1633-1642 (1996)).

This disclosure provides a 3D microenvironment surface to activate at least two different receptors simultaneously by presenting a substrate protein having combinatorial motifs comprising at least two different peptide motifs that bind to at least two different receptors, respectively. The suitable combinatorial motifs may include one or more spacers between two peptide motifs to optimize flexibility and/or solubility and so afford increased affinity and/or bioavailability. The combinatorial motifs may have a peptide spacer sequence of at least two amino acids, preferably 2-15 amino acids, appended to the C-termini of at least one of the two peptide motifs.

In one embodiment of the disclosure, a 3D microenvironment surface that combinatorially regulates the activity of both integrin and growth factor receptor to support self-renewal and pluripotency of murine embryonic stem cell is provided. The microenvironment surface comprises mussel adhesive protein as a substrate protein, functionalized with two peptide motifs; one is fibronectin-derived peptide PHSRN-GRGDSP (SEQ ID NO:47) to target α5β1 and the other is FGF2-derived peptide ANRYLAMKEDGRLLAS (SEQ ID NO:33) to target FGF receptor, FGFR2IIIc.

In one embodiment of this disclosure, a combinatorial 3D microenvironment surface comprising a nanofiber substrate having an average diameter of 100 nm to 20 microns, wherein the nanofiber surface presents extracellular components comprising extracellular matrix mimetic, growth factor mimetic, WNT mimetic, cytokine mimetic such as IL-3, LIF mimetic or combinations thereof.

This disclosure can be used in high throughput screening (HTS) to identify combinatorial surface ligands to engineer optimal synthetic microenvironment that can specifically, selectively, simultaneously or sequentially generate signaling pathway to regulate self-renewal and pluripotency of pluripotent stem cells.

A “microenvironment array” is a combination of two or more microlocations. Preferably, an array is comprised of microlocations in addressable rows and columns. The layout of microenvironment arrays produced according to the disclosure can vary, dependent upon the particular pluripotent stem cell lines.

The disclosure provides for a device of microenvironment array comprising:
(a) preparing biochemical cue composition;
(b) placing the composition on surface of a substrate for coating; and
(c) obtaining the extracellular microenvironment array.

In one embodiment of this disclosure, a microenvironment array is provided. The array is a 12-well, microwell plate consisting of 4×3-well. Each well within a strip (4 wells total) is pre-coated with a different biofunctional composition to generate different extracellular microenvironment. Cells of interest can be seeded onto each well, whereby cells are cultured on different extracellular microenvironment surfaces. An extracellular microenvironment that induces a desirable cellular behavior can be identified and designed from the assay utilizing this extracellular microenvironment array.

The following examples are provided to demonstrate preferred embodiments of this disclosure and the disclosure is not intended to be limited in scope by the specific embodiments described herein, which are intended for the purposes of exemplification only. Functionally equivalent products, compositions and methods are clearly within the scope of the disclosure, as described herein.

### EXAMPLES

### Example 1

### Preparation of Electrospinnable Biofunctional Composition to Engineer an Extracellular Microenvironment

PVDF with an average molecular weight of 200 kDa from SigmaAldrich (St. Louis, USA), PAN with an average molecular weight of 200 kDa, PES with an average molecular weight of 200 kDa purchased from SigmaAldrich (St. Louis, USA), PLA with an average molecular weight of 200 kDa purchased from SigmaAldrich were dissolved in DMAc to prepare 20 wt % solution. MAPTRIX® ECM purchased from Kollodis BioSciences (North Augusta, S.C., USA) was dissolved in an aqueous solution composed of distilled water and DMAc. Each polymer solution was mixed well, together with MAPTRIX® ECM solution, by vortexing it for 10 minutes to make homogenous 18 wt % solution.

The electrospinnable solution was placed in a plastic syringe fitted with a 27 G needle. A syringe pump (KD Scientific, USA) was used to feed the polymer solution into the needle tip. A high voltage power supply was used to charge the needle tip. The nanofibers were collected onto grounded aluminum foil target located at a certain distance from the needle tip. The fiber meshes were then removed, placed in a vacuum chamber for two days to remove residual solvent, and then stored in a desiccator.

The electrospinnable composition and electrospinning conditions are summarized in Tables 1 and 2, respectively.

TABLE 1
Electrospinnable solution composition (structural component)

| Polymer | Solvent | MAPTRIX ® | Solvent (1 mL) |
|---|---|---|---|
| PVDF 100 mg | DMAc 5 mL | 7 mg | DW/DMAc (0.1/0.9) |
| PES 100 mg | DMAc 5 mL | 7 mg | DW/DMAc (0.1/0.9) |
| PAN 100 mg | DMAc 5 mL | 7 mg | DW/DMAc (0.1/0.9) |
| PLGA 100 mg | DMAc 5 mL | 7 mg | DW/DMAc (0.1/0.9) |
| PVDF/PAN | DMAc 5 mL | 7 mg | DW/DMAc (0.1/0.9) |
| 50 mg/50 mg | | | |
| PVDF/PES | DMAc 5 mL | 7 mg | DW/DMAc (0.1/0.9) |
| 50 mg/50 mg | | | |

TABLE 2
Electrospinning Parameters

| | | | | Distance |
|---|---|---|---|---|
| E-Solution | Concentration | Voltage (kv) | Rate (mL/min.) | (cm) |
| PVDF | 18% | 21 | 0.1 | 11 |
| PES | 18% | 21 | 0.1 | 10 |
| PAN | 18% | 21 | 0.1 | 10 |
| PLGA | 18% | 21 | 0.1 | 11 |
| PVDF/PAN | 18% | 21 | 0.1 | 10 |
| PVDF/PS | 18% | 21 | 0.1 | 10 |

E-solution is the electrospinnable biofunctional composition prepared from the procedure described above in Example 1.

### Example 2

### Preparation of Nanofiber Having Different Diameter

Each Polyvinylidene fluoride (PVdF)-Kynar 761(Homopolymer, Mw: 400,000-500,000), and Polyvinylidene fluoride (PVdF)-Solef 21216(Co-polymer, Mw: 600,000) or Polyacrylonitril-Pulver(Co-PAN, Mw: 85,000) was dissolved in DMAC and blended. The blending ratio of homopolymer to copolymer were 1:1, 1:2, 1:3, 1:4, 1:5, 1:6, 1:7, 1:8, 1:9. Nanofibers having different diameter were formed by the same procedure mentioned in Example 1.

### Example 3

### Preparation of Nanofiber Having Particles

MAPTRIX® ECM-based particles were formed by reaction of the carboxyl group of MAPTRIX® activated with 1-ethyl-3-(3-dimethylaminopropyl) carbodiimides/N-hydroxysulfosuccinimide (EDC/s-NHS) on the C-terminus with the amino groups of the MAPTRIX®.

1-Ethyl-3-[3-Dimethylaminopropyl]carbodiimide hydrochloride (EDC) solution is prepared by dissolving 10 mg of EDC in 1 ml of sodium bicarbonate buffer (10 mM, pH 6.5). 5 mg of solid sulfo-N-hydroxysulfosuccinimide (S-NHS) is added to the EDC solution. The EDC/S-NHS solution is added to the nanofiber surface to activate carboxyl group on the nanofiber surface for 30 minutes. After the C-terminus activation, 0.1 mg of MAPTRIX® having PHSRN-RGDSP (SEQ ID NO:17) motif dissolved in 1 mL distilled water was added to the nanofiber surface. Cross-linking is carried out at ambient temperature for 30 minutes to get cross-linked MAPTRIX® particle-presenting PHSRN-RGDSP (SEQ ID NO:17) on the nanofiber surface.

As presented in FIG. 3, the MAPTRIX® particles were observed. Depending on the concentration of MAPTRIX®, the particle size ranged from 0.5 to 5 μm.

### Example 4

### Microenvironment Array Preparation

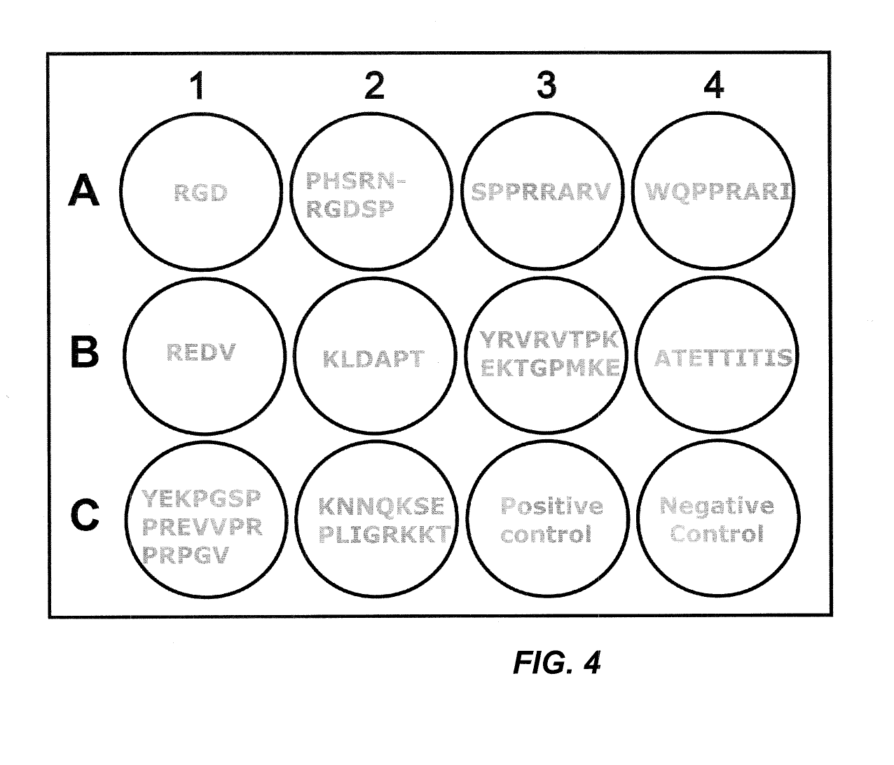

Several arrays of twelve different extracellular microenvironments were prepared as represented in FIGS. 2 and 4. A representative array surface to present fibronectin-derived peptide motif to bind integrin, heparin and sydecan and was screened to identify microenvironmental surfaces that promote self-renewal and pluripotency of murine embryonic stem cell.

For extracellular microenvironment array, stock solutions of each ECM and GF mimetic were suspended and dissolved in distilled water at 0.06 mg/mL. ECM and/or GF mimetic solutions were then used in single or mixed in 12 different combinations in a 12-microwell plate. The layout for each extracellular microenvironment array was represented in FIGS. 2 and 4, respectively.

For single or combinatorial microenvironment array preparation, MAPTRIX® containing integrin binding motif such as RGD (SEQ ID NO:15), GRGDSP (SEQ ID NO:16), PHSRN-RGDSP (SEQ ID NO:17), or KLDAPT (SEQ ID NO:43) and heparin binding motif such as SPPRRARVT (SEQ ID NO:18), WQPPRARI (SEQ ID NO:19), KNNQKSEPLIGRKKT (SEQ ID NO:20), ATETTITIS (SEQ ID NO:48), or YEKPGSPPREVVPRPRPGV (SEQ ID NO:49) were used to create extracellular microenvironment surface in each well.

### Example 5

### Culture and Self-Renewal of ESCs on Biochemically Defined Surfaces Generating Signaling Pathway for Self-Renewal and Pluripotency

The ability of an extracellular microenvironment surface to support self-renewal of mESCs was evaluated by serial passaging of murine ES cells on the microenvironment surface as prepared in Example 1. These murine ES cells were obtained from cultures of early blastocysts.

The array was incubated with media-containing serum replacement media and murine embryonic stem cells were grown on the array for 5 days. For the maintenance of murine embryonic stem cell cultured on poly-D-lysine (PDL, Sigma-Aldrich) coated surface, DMEM Glutamax (GIBCO, Life Technology) containing high glucose 4.5 g/L, Na-pyruvate (0.11 g/L) and L-glutamine was used with 1% non-essential amino acid (Sigma-Aldrich), 50 U/mL Penicillin/streptomycin (GIBCO) and 0.1 mM 2-Mercaptoethanol (GIBCO) as the basal medium, which was added with 20% fetal bovine serum (FBS, Hyclone) and leukemia inhibitory factor (LIF, 1,000 units/mL, Millipore) at 37° C., 5% CO2 incubator.

The mESCs was cultured in KnockOut™ DMEM medium (Invitrogen) supplemented with 20% KnockOut™ Serum Replacement (KSR; Invitrogen), 0.1 mM of 2-mercaptoethanol (Invitrogen), MEM Non-essential Amino Acids (Invitrogen), GlutaMAX™ Supplement (Invitrogen), leukemia inhibitory factor (LIF, 1,000 units/mL, Millipore), and 20 ng/mL of MAPTRIX® PHSRN-RGDSP (SEQ ID NO:17) (Kollodis BioSciences).

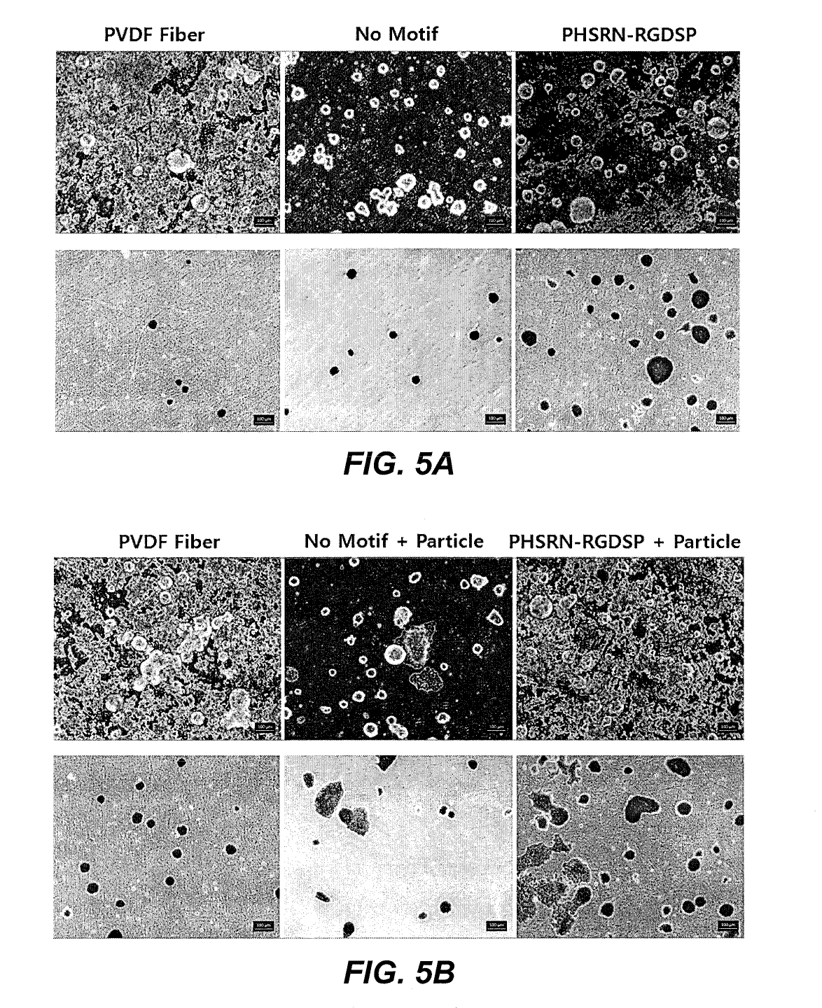

To elucidate the effect of microenvironmentally defined surface on self-renewal and pluripotency of murine embryonic stem cells, the cells (6×10⁴) were cultured for 96 hours on twelve different microenvironment array as represented in FIGS. 2 and 5.

The embryonic stem cells were monitored by using an alkaline phosphatase staining (FIGS. 5A and 5B) and it was confirmed that the murine embryonic stem cells were maintained in an undifferentiated stage on the surface-presenting integrin binding peptide motif.

What is claimed is:

1. An electrospinnable biofunctional composition to engineer 3D extracellular microenvironment comprising two components, an extracellular component and a structural component.

2. A nanofiber having particles presenting peptide motifs to activate at least one cell surface receptor.

3. The nanofiber of claim 2, wherein at least one cell surface receptor is selected from the group consisting of integrin, growth factor receptor, heparin, syndecan, and a combination of any thereof.

4. A microenvironment surface presenting at least one ECM-derived peptide motif that regulates cellular behavior.

5. The microenvironment surface of claim 4, wherein the cellular behavior is selected from the group consisting of cell adhesion, migration, growth, differentiation, and a combination of any thereof.

6. A process for producing a microenvironment array, the process comprising:
placing an electroprocessable biofunctional composition on a solid support in a pattern; and
electroprocessing the composition to obtain an extracellular microenvironment array.

7. An extracellular microenvironment surface for long-term self-renewal of a pluripotent stem cell, wherein the extracellular microenvironment surface presents signal molecules that support the pluripotent stem cell.
